(19) United States
(12) Patent Application Publication
(10) Pub. No.: US 2003/0055332 A1
(43) Pub. Date: Mar. 20, 2003

(54) MRI compatible guidewire

(76) Inventors: Wolfgang Daum, Groton, MA (US); Axel Winkel, (US); Guido Pantel, Gorslow (DE)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 09/954,720

(22) Filed: Sep. 14, 2001

(51) Int. Cl. A61B 5/05

(52) U.S. Cl. 600/420; 604/530

(57) ABSTRACT

MRI compatible device for the guiding of catheters in human or animal vessels.

### PROBLEM

With the aid of contrast media, MRI devices are suitable for the imaging of human vessels, especially in angiography. To maneuver balloons, stents, or other devices inside the vessel, however, catheters with guide wires are necessary. A guide wire is a long piece of wire with a pre-bent tip, used to conduct or guide catheters through vessels. Due to its metallic characteristics, a guide wire forms an electric conductor that can be heated by current or voltage induction upon application under MRI owing to the high-frequency and magnetic field, with the danger of the patient being locally burned.

The invention presented herein aims at solving this problem.

### SOLUTION

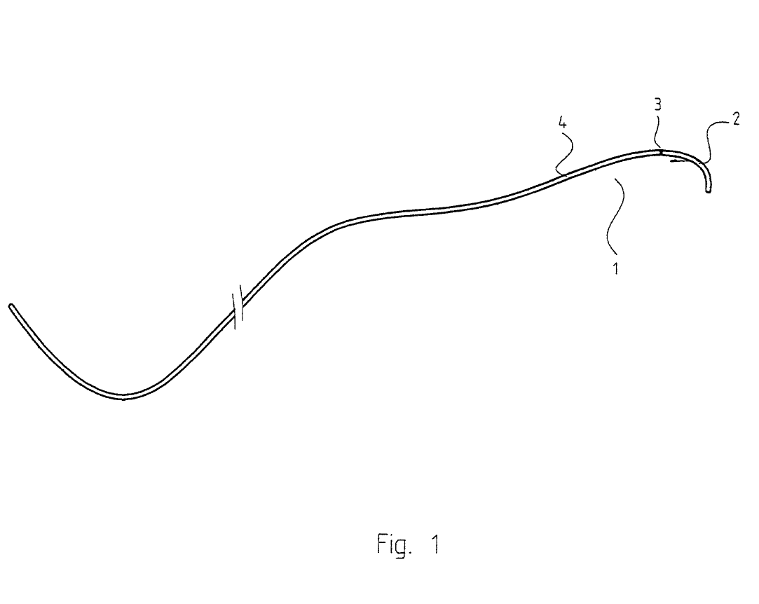

The solution of the problem is shown in FIG. 1.

FIG. 1 Axial section through the invented guide wire

FIG. 1 shows an axial section of the invented guide wire, consisting of a distal metallic part 2 and an MRI-inert main part 4 as well as a connection 3 between both parts. The metallic distal part, which can be pre-bent by 90° as shown in the figure, serves as a sufficiently rigid and thus guidable piece to guide the wire through the vessels into defined vascular shunts. The main part 4 of the guide wire needs to be designed to solely carry and transmit the pressure applied from proximal towards distal for the advance of the metallic distal part 2 attached by the connection 3. The distal part 2 is also essential as it can be imaged due its susceptibility artifact. The wire dimensions need to be designed in a way that the distal part provides sufficiently small artifacts that do not obscure the vessels. The elasticity may be the same as with normal, MRI incompatible guide wires designed for x-ray fluoroscopy or x-ray CT, using, for example, nickel-titanium or another flexible titanium alloy.

Due to the short length of this distal part 2, the current or voltage induced on this part will be low enough to incur neither a considerable overheating of the surrounding tissue nor local vascular burns. Such distal part 2 is, therefore, approx. 5 to 15 cm, typically 8 to 10 long with a total catheter length of 60 to 200 cm. The diameter of the distal part 2 varies between 0.1 mm (neuro applications) and 1.5 mm (large leg vessels), typically between 0.6 and 0.8 mm.

The long main part 4 of the guide wire is manufactured of an artificial material supporting and transmitting the guiding pressure from proximal to distal. The main part 4 is designed of plastics so that neither current nor voltage is induced. This main part 4 is an insulator or has a very high electric resistance, preventing any overheating and thus local burn effects for the patient.

Such guide wire can be inserted for guidance into conventional catheters. The main part may have a length between 40 and 200 cm; typically it will be between 60 and 80 cm long.

The connection 3 can be designed in different ways. It could be a gluing, or the main part 4 is pinched over or under the distal front part 2 at the connection 3. Screwing connections would also be feasible, as well as a diminution of the main part at its front end which could then lead into the interior of the distal part 2 and be glued or pinched therein.

MRI compatible device for the guiding of catheters in human or animal vessels.

Names

1. Guide wire

2. Distal part

3. Connection

4. Main part of the guide wire

1. An MRI compatible device for guiding catheters inside human or animal vessels, comprising a metallic wire distal part and an MRI-inert plastics main part.

2. The device according to claim 1, wherein the metallic distal part comprises nickel-titanium.

3. The device according to claim 1, wherein the main part comprises an artificial material.

4. The device according to claim 1, wherein the main part and the distal part are glued together.

5. The device according to claim 1, wherein the distal part is pinched onto the main part.

6. The device according to claim 1, wherein the main part and the distal part are connected by shrinkdown plastic tubing.

7. The device according to claim 1, wherein the main part comprises a core in a center of the main part.

8. The device according to claim 1, wherein the metallic distal part comprises stainless steel alloy.

9. The device according to claim 3, wherein the artificial material is selected from the group consisting of polypropylene (PP), polyethylene (PE), polyetherimides (PEI), and polyetheretherketone (PEEK).

10. The device according to claim 7, wherein the core comprises an insulant material.
